(19) United States
(12) Patent Application Publication
(10) Pub. No.: US 2023/0263802 A1
(43) Pub. Date: Aug. 24, 2023

(54) METHODS OF REDUCING INCIDENCE OR RISK OF CEREBRAL FOLATE DEFICIENCY

(71) Applicants: AYOUB, George, Santa Barbara, CA (US); LAM, Dominic Man-Kit, Wan Chai (CN)

(72) Inventors: George Ayoub, Santa Barbara, CA (US); Dominic Man-Kit Lam, Wan Chai (CN)

(21) Appl. No.: 18/016,006

(22) PCT Filed: Jul. 27, 2021
(86) PCT No.: PCT/IB2021/056815

§ 371 Date: Jan. 13, 2023

Related U.S. Application Data
(60) Provisional application No. 63/056,829, filed on Jul. 27, 2020.

(51) Int. Cl. A61K 31/519; A61K 45/06

(52) U.S. Cl. CPC A61K 31/519; A61K 45/06

(57) ABSTRACT

Methods of reducing incidence of or risk of developing a cerebral folate deficiency (CFD) or a CFD-related disorder comprising administering reduced folate compounds.

### BACKGROUND

Vitamin B-9 (folate) is important for fetal brain development. Low levels of Vitamin B-9 are known to trigger depression in certain people. To protect fetal brain development, in the United States, Vitamin B-9 in the form of folic acid has been added to foods since the 1990s, so it is rare to have insufficient amounts of this vitamin in the blood.

In some people, folate is present in the blood but cannot be absorbed into the brain at appropriate levels. This condition, called cerebral folate deficiency (CFD), interferes with normal brain development and normal brain function, and is linked to mental health disorders such as Autism Spectrum Disorder (ASD) and depression.

Often, CFD stems from insufficient levels of folates in the blood crossing the blood brain barrier (BBB). To make folate available to the brain, the body converts folate from an oxidized form (folic acid) to a reduced form (methyl-folate). While the folate added to foods is oxidized, the folate that the brain absorbs is reduced. Processing from folic acid to methyl-folate typically occurs in the digestive system.

In some people, a genetic variant in a gene called MTHFR (methylen-tetra-hydro-folate reductase), and/or a variant of the gastrointestinal tract microbiome, leads to this inability to reduce folate sufficiently, making it difficult for the folate to cross the blood-brain barrier.

A separate issue impacting folate transfer across the blood-brain barrier is the presence, in the blood of some individuals, of an antibody against the folate receptor that carries folate across the blood-brain barrier. This antibody blocks the more efficient folate transporter (the “high-affinity transporter”), leaving only the lower efficiency folate transporter (“the low-affinity transporter”). The low-affinity transporter needs extra folate in the blood to transport enough into the brain. Those with the folate receptor antibody thus need higher levels of reduced folate in the blood to navigate this low-affinity transporter, necessitating the supplementation with methyl-folate or L-folinic acid as stated in our claims.

There remains a need for improved methods of treatment, reducing incidence, and/or reducing risk of developing CFD-related disorders. The problem is solved by compositions as defined in claim 1. Preferred embodiments are subject to the dependent claims. The problem is solved according to methods disclosed.

### SUMMARY

The present disclosure relates to methods and kits for reducing incidence of, or reducing risk of developing, cerebral folate deficiency (CFD) or a CFD-related disorder.

In one aspect, the invention relates to methods of reducing incidence or reducing risk of developing cerebral folate deficiency (CFD) or a CFD-related disorder, the methods comprising the step of administering an effective amount of a reduced folate compound to a woman of child-bearing age, wherein (a) the woman or a sexual partner of the woman has a family history of a CFD-related disorder, (b) one or more FRα autoantibodies has been detected in a fluid sample from the woman or a sexual partner of the woman, and/or (c) the woman or a sexual partner of the woman (i) has a mutation in the methylenetetrahydrofolate reductase (MTHFR) gene or (ii) has a proximate family member with a mutation in the MTHFR gene.

In one aspect, provided are methods of reducing incidence of cerebral folate deficiency (CFD) or a CFD-related disorder, the methods comprising the step of: administering an effective amount of a reduced folate compound to a woman of child-bearing age, wherein: (a) the woman has not been determined to be pregnant; and (b) (i) the woman or a sexual partner of the woman has a family history of a CFD-related disorder; (ii) one or more FRα autoantibodies has been detected in a fluid sample from the woman or a sexual partner of the woman; or the woman or a sexual partner of the woman (1) has a mutation in the methylenetetrahydrofolate reductase (MTHFR) gene or (2) has a proximate family member with a mutation in the MTHFR gene.

In one aspect, provided are methods of reducing risk of a fetus developing cerebral folate deficiency (CFD) or a CFD-related disorder, the methods comprising the step of: administering an effective amount of a reduced folate compound to a pregnant woman bearing the fetus, wherein the pregnant woman, the fetus, or the fetus's biological father has a family history of a CFD-related disorder. In some embodiments, one or more FRα autoantibodies has been detected in a fluid sample from the pregnant woman, the fetus, or the fetus's biological father. In some embodiments, the fetus or the pregnant woman (i) has a mutation in the methylenetetrahydrofolate reductase (MTHFR) gene or (ii) has a proximate family member with a mutation in the MTHFR gene.

In one aspect, provide are methods of reducing risk of a child developing cerebral folate deficiency (CFD) or a CFD-related disorder, the method comprising the step of: administering an effective amount of a reduced folate compound to a woman who is breastfeeding the child. In some embodiments, the child or a biological parent of the child has a family history of a CFD-related disorder. In some embodiments, one or more FRα autoantibodies has been detected in a fluid sample from the child or a biological parent of the child. In some embodiments, the child (i) has a mutation in the methylenetetrahydrofolate reductase (MTHFR) gene or (ii) has a proximate family member with a mutation in the MTHFR gene.

In one aspect, provided are methods of reducing risk of a subject developing cerebral folate deficiency (CFD) or a CFD-related disorder, the methods comprising the step of: administering an effective amount of a reduced folate compound to the subject, wherein the subject has a family history of a CFD-related disorder. In some embodiments, one or more FRα autoantibodies has been detected in a fluid sample from the subject, the subject's biological mother or the subject's biological father. In some embodiments, the subject (i) has a mutation in the methylenetetrahydrofolate reductase (MTHFR) gene or (ii) has a proximate family member with a mutation in the MTHFR gene. In some embodiments, the subject is a child, for example, a child of less than 3 years of age, less than 2.5 years of age, less than 2 years of age, less than 1.5 years of age, less than 1 year or age, or less than 6 months of age.

In some embodiments, provided methods further comprise co-administering an effective amount of one or more additional agents selected from vitamin B3, vitamin B12 (methylcobalamin), vitamin D3, and a subunit of FRα. For example, in some embodiments, an effective amount of vitamin B12 and an effective amount of vitamin D3 are co-administered with the effective amount of the reduced folate compound.

In some embodiments, the CFD-related disorder is selected from the group consisting of autism spectrum disorder, obsessive compulsive disorder, attention deficit/hyperactivity disorder, and depression.

In some embodiments, the fluid sample comprises a blood sample, for example, a plasma or serum sample, a whole blood sample, or a cellular fraction of a whole blood sample. In some embodiments, the fluid sample comprises an amniotic fluid, ascites, cerebrospinal fluid, lymph, sweat, urine, tears, saliva, pleural fluid, pericardial fluid, cavity rinse, or organ rinse sample.

In some embodiments, the reduced folate compound is selected from the group consisting of folinic acid, methyltetrahydrofolate (MTHF), folinic acid calcium salt, leucovorin, levo-leucovorin calcium, a mixture of dextra-leucovorin and levo-leucovorin, and combinations thereof. In some embodiments, the reduced folate compound is folinic acid or MTHF.

In some embodiments, the step of administering comprises oral administration. For example, the reduced folate compound may be administered as a food additive or a tablet. In some embodiments, the tablet comprises the reduced folate compound in lyophilized form.

In some embodiments, the step of administering comprises administering the reduced folate compound intranasally.

In one aspect, provided are kits comprising one or more pharmaceutically acceptable dosage forms, wherein at least one pharmaceutically acceptable dosage form comprises a reduced folate compound, and instructions for use according to methods provided herein. In some embodiments, the one or more pharmaceutically dosage forms collectively comprise one or more additional agents selected from the group consisting of vitamin B12 (methylcobalamin) and vitamin D3. In some embodiments, the one or more pharmaceutically dosage forms collectively comprise vitamin B12 and vitamin D3.

### DETAILED DESCRIPTION

The present invention encompasses the recognition that some cerebral folate deficiency (CFD)-related disorders are diagnosed and treated only after a critical window for intervention has already passed. For example, behavioral symptoms of autism spectrum disorder (ASD) first appear at about ages 3 to 4, when it may be too late to prevent ASD from being reversed or treated fully. Moreover, behavioral testing to diagnose ASD is time-consuming and expensive, and may further delay interventions that may have been successful, if introduced earlier.

Additionally, existing interventions for folate deficiencies are inadequate to address problems specific to cerebral folate deficiencies. For example, pregnant women are recommended to take folic acid supplements. However, folic acid is not transported across the blood-brain barrier. Other existing interventions may not intervene early enough or may miss certain groups of potentially at-risk subjects.

In some embodiments, methods disclosed herein overcome these and other problems by administering chemically reduced folate compounds (which, in contrast to folic acid, are transported across the blood-brain barrier) to various subjects (e.g., women of childbearing, pregnant women, breastfeeding women, or children), in order to reduce the incidence or risk of CFD-related disorders at early developmental periods, such as during fetal development and early childhood. Methods disclosed herein may, in some embodiments, circumvent effects of folate receptor alpha antibodies (FRAA) that interfere with folate transport into the brain.

### DEFINITIONS

As used herein, the term “cerebral folate deficiency” (CFD) generally refers to a condition characterized by low concentrations of 5-methyltetrahydrofolate (MTHF) in the cerebrospinal fluid in the presence of normal or undefined blood folate concentrations. CFD may result from, e.g., lack of ability of folate in the blood to cross the blood-brain barrier (BBB), such as occurs with certain variants and/or deficiencies of gene products involved in folate transport and/or folate metabolism (e.g., folate reduction). For example, deficiencies and/or variants in folate receptor alpha (FRα), dihydrofolate reductase (DHFR), methylenetetrahydrofolate reductase (MTHFR), and/or methenyltetrahydrofolate synthetase (MTHFS) are known as causes of CFD. Additional conditions that are known to cause CFD include folate receptor alpha antibodies (FRAAs) in the blood, mitochondrial disorders, serine deficiency, dihydropteridine reductase (DHPR) deficiency, and aromatic 1-amino acid decarboxylate (AADC) deficiency.

As used herein, the terms “cerebral folate deficiency-related disorder” or “CFD-related disorder” are used interchangeably and refer to any of a variety of conditions that are associated with CFD. In some embodiments, the CFD-related disorder is characterized by one or more symptoms selected from the group consisting of ataxia, autistic features, dyskinesia, hearing loss, hypotonia, seizures, spasticity, vision loss, and combinations thereof. A disorder associated with CFD may be generally referred to as “CFD syndrome.” Examples of disorders that may be associated with CFD include, but are not limited to, attention deficit/hyperactivity disorder, autism spectrum disorder (ASD), bipolar disorder, epilepsy, depression, obsessive compulsive disorder, and spina bifida.

As used herein, the term “effective amount” refers to an amount sufficient, at dosages and for periods of time necessary, to achieve a desired result, e.g., reduced incidence of and/or risk of developing a CFD-related disorder. An “effective amount” may depend on the context in which it is being applied and the specific disorder whose incidence or risk is being lowered. An “effective amount” may be administered in a single dose or in multiple (e.g., at least two, at least three, at least four, at least five) doses. In some embodiments, an “effective amount” is administered via regular doses (e.g., thrice daily, twice daily, daily, every two days, every three days, twice a week, once a week, once every two weeks, etc.).

As used herein, the term “folate receptor alpha,” abbreviated “FRα,” refers to the alpha isoform of the folate receptor, a glycosylphosphatidylinositol (GPI-anchored membrane protein with high affinity for binding and transport of the active form of folate, 5methyltetrahydrofolate (5-MTHF). FRα is also known as FOLR1 or folate binding protein.

As used herein, the term “folate receptor alpha antibody,” abbreviated FRAA and also known as “folate receptor alpha autoantibody,” refers to an antibody that is capable of binding to FRα. In some embodiments, a FRAA impairs folate transport into the brain. For example, a “blocking FRAA” directly interferes with the binding of folate to FRα. For example, a “binding FRAA” triggers an antibody-mediated immune reaction upon binding to FRα.

As used herein, the term “folate-binding fragment” refers to a fragment of a folate-binding molecule (e.g., a folate receptor, such as FRα), which fragment is capable of binding to folate.

### Methods of Reducing Incidence or Risk of CFD or a CFD-Related Disorder

Provided methods generally comprise a step of administering an effective amount of a reduced folate compound to a subject when one or more situations (as elaborated further herein) apply.

For example, provided are methods of reducing incidence of CFD or a CFD-related disorder by administering an effective amount of a reduced folate compound to a woman of child-bearing age, wherein one or more situations with respect to the woman or a sexual partner of the woman applies. In some embodiments, the woman is not determined to be pregnant. In some such embodiments, the woman is planning to become pregnant.

Also provided are methods of reducing risk of a fetus developing CFD or a CFD related disorder by administering a reduced folate compound to a pregnant woman, when one or more situations with respect to the pregnant woman, the fetus, and/or the fetus's biological father apply.

Also provided are methods of reducing risk of a child developing CFD or a CFD-related disorder by administering a reduced folate compound to (1) a woman who is breastfeeding the child or (2) the child, when one or more situations regarding the child, the child's biological mother, or the child's biological father apply.

In various methods disclosed herein, one or more of the following situations may indicate that a subject should be administered a reduced folate compound in accordance with methods disclosed herein: (1) family history of a CFD-related disorder; (2) detection of FRα autoantibodies in a fluid sample; and/or (3) presence of a genetic marker associated with increased risk of developing a CFD-related disorder.

### Family History

In accordance with some methods of the present disclosure, a subject may be administered reduced folate compounds to reduce incidence of a CFD-related disorder or to reduce risk of a fetus or child developing a CFD-related disorder when a relevant individual (e.g., the subject, a sexual partner of the subject, the fetus, the child, or any biological parent of the fetus or child) has a family history of the CFD-related disorder.

Generally, an individual has a family history of a CFD-related disorder when a proximate family member (that is, a biological parent, a biological grandparent, a biological child, a sibling sharing at least one biological parent, a sibling of a biological parent, or a first cousin) of that individual is diagnosed as having a CFD-related disorder. The proximate family member may be diagnosed with the same or different CFD-related disorder than the CFD-related disorder whose incidence is reduced, or for which the risk of developing is reduced, by the methods disclosed herein. For example, in some embodiments, provided methods reduce incidence of or risk of developing of autism spectrum disorder (ASD), wherein a concerned individual has a family history of ASD. As another example, in some embodiments, provided methods reduce incidence of or risk of developing ASD, wherein a concerned individual has a family history of a different CFD-related disorder (e.g., attention deficit/hyperactivity disorder, bipolar disorder, epilepsy, depression, obsessive compulsive disorder, and spina bifida).

### Detection of FRα Autoantibodies

In accordance with some methods of the present disclosure, a subject may be administered reduced folate compounds to reduce incidence of a CFD-related disorder or to reduce risk of a fetus or child developing a CFD-related disorder when one or more FRα autoantibodies has been detected in a fluid sample from a relevant individual (e.g., the subject, a sexual partner of the subject, the fetus, the child, or any biological parent of the fetus or child). 20 The fluid sample may comprise a blood, amniotic fluid, ascites fluid, cerebrospinal fluid, lymph, sweat, urine, tears, saliva, pleural fluid, pericardial fluid, cavity rinse, or organ rinse samples, or a mixture of any of the foregoing. Examples of suitable blood samples include, but are not limited to, plasma samples, serum samples, whole blood samples, cellular fractions of whole blood samples, and mixtures of any of the foregoing.

Methods of detecting FRα autoantibodies (FRAs) include methods known in the art and methods described herein. In some embodiments, the method is a quantitative or semiquantitative method.

In some embodiments, an enzyme-linked immunosorbent assay (ELISA) (e.g., a direct ELISA, an indirect ELISA, or a sandwich ELISA) is used to detect FRα autoantibodies.

30 For example, in one version of a suitable indirect ELISA format, 1) FRα (or a folate-binding fragment thereof) is immobilized on the surface of vessels (e.g., wells), 2) samples are added to the vessels and incubated (FRA in the sample may bind to the immobilized FRα (or a folate-binding fragment thereof), 3) labeled antibodies against FRA are added and incubated with the samples, and 4) label is measured. More measured label indicates more FRA in the sample.

In a competitive version of the above-described indirect ELSA, labeled folic acid is used instead of labeled antibodies against FRA in step 3) and more measured label indicates less FRA in the sample.

In one version of a suitable sandwich ELISA format, 1) antibodies against FRA is immobilized on the surfaces of vessels (e.g., wells), 2) samples are added to the vessels and incubated (FRA in the sample may bind to the immobilized antibodies against FRA), 3) labeled antibodies against FRA are added and incubated with the samples (thereby possibly forming a sandwich of immobilized antibodies against FRA, FRA from the sample, and labeled antibodies against FRA) and 4) the label is measured. More measured label indicates more FRA in the sample. A competitive version of such a sandwich ELISA assay, in which less measured label 15 indicates more FRA in the sample, may also be used.

In some embodiments, a competitive radioimmunoassay (RIA) may be used to detect FRα autoantibodies. For example, in one version of a competitive RIA to detect FRAs, fluid samples are added to a vessel (e.g., a well) having a surface coated with FRα (or a folate binding fragment thereof). Radiolabeled folate receptor antibodies (FRAs) are then added and then incubated, then the vessel is washed. The radiolabeled FRA competes with any FRA in the sample for binding to FRα (or a folate-binding fragment thereof) coated on the vessel's surface. Radioactivity is measured, and decreased radioactivity indicates more FRA in the sample. A similar competitive RIA may be used in which antibodies against FRAs are used in place of the FRα (or a folate-binding fragment thereof) to coat the surface of the vessel.

In some embodiments, a lateral flow immunoassay is used to detect FRα autoantibodies. For example, in one version of such an assay, an absorbent strip contains, in the following order (1) a conjugate pad comprising gold-tagged FRα (or a folate-binding fragment thereof) and gold-tagged control antigen; (2) a first strip on which antibodies against FRAs are immobilized; and (3) a second strip on which control antibodies are immobilized. During the assay, the sample is flowed past the conjugate pad, and any FRAs in the sample may bind to the gold-tagged FRα (or a folate-binding fragment thereof), thereby forming complexes of FRA in the sample and gold-tagged FRα (or a folate-binding fragment thereof), while control antigens form the conjugate pad are caught up the flow. The sample is flowed past the first strip, where any complexes that form are caught by the immobilized antibodies against FRA. The first strip becomes colored if there is enough FRA in the sample. This sample is then flowed past the second strip, where gold-tagged control antigens originally from the conjugate pad may bind to the immobilized control antibodies. The second strip becomes colored if the assay worked.

Other versions of lateral flow assays may also be suitable. For example, (i) in the conjugate pad, gold-tagged antibodies against FRA may be used in place of the gold-tagged FRα (or a folate-binding fragment thereof), and (ii) in the first strip, FRα (or a folate-binding fragment thereof) may be used in place of antibodies against FRA.

### Genetic Markers

In accordance with some methods of the present disclosure, a subject may be administered reduced folate compounds to reduce incidence of a CFD-related disorder or to reduce risk of a fetus or child developing a CFD-related disorder when a relevant individual (e.g., the subject, a sexual partner of the subject, the fetus, the child, or any biological parent of the fetus or child) (i) has a genetic marker associated with increased risk of developing a CFD related disorder or (ii) has a proximate family member who has a genetic marker associated with 20 increased risk of developing a CFD-related disorder.

In some embodiments, the genetic marker is associated with a mutation or variant that affects the function of a gene product involved in folate reduction, folate transport, and/or folate metabolism, for example, folate receptor alpha (FRα), dihydrofolate reductase (DHFR), methylenetetrahydrofolate reductase (MTHFR), methenyltetrahydrofolate synthetase (MTHFS), dihydropteridine reductase (DHPR), and aromatic 1-amino acid decarboxylate (AADC). In some embodiments, the genetic marker is associated with a mutation in the methylenetetrahydrofolate reductase (MTHFR) gene.

### Reduced Folate Compounds

As used herein, the term “reduced folate compound” generally refers to a folate compound that is reduced folic acid (the oxidized form of folate). Such reduced forms of folate are generally more metabolically active than folic acid. For example, folinic acid is more metabolically active than folic acid and is an immediate precursor to 5,10-methylenetetrahydrofolate, which in turn is rapidly metabolized to produce active folate (5methyltetrahydrofolate (5-MTHF)). 5-MTHF is able to cross the blood-brain barrier.

Suitable reduced folate compounds include, but are not limited to, folinic acid (e.g., L-folinic acid), 5-MTHF, and analogs, derivatives, mixtures, or combinations thereof. For example, leucovorin (a racemic mixture of the diastereoisomers of the 5-formyl derivative tetrahydrofolic acid), levo-leucovorin (a pharmacologically active isomer of leucovorin), and/or mixtures of dextra-leucovorin and levo-leucovorin, may be used as reduced folate compounds. Derivatives, metabolites, prodrugs, stereoisomers, polymorphs, analogues, and/or pharmaceutically acceptable salts of any of the foregoing may also be suitable. For example, folinic acid calcium, leucovorin calcium, and levoleucovorin calcium may be used.

In some embodiments, administering folinic acid or an analog or derivative thereof bypasses the need for dihydrofolate reductase (DHFR) activity. In some embodiments, administering 5-MTHF or an analog or derivative thereof bypasses the need for methylenetetrahydrofolate reductase (MTHFR) or DHFR.

### Additional Agents

Additional agents may also be co-administered with reduced folate compounds. By “co-administered” (a term that may be used interchangeable with “in combination with”), it is meant that the reduced folate compound and the additional agent may be administered in the same composition or in separate compositions (e.g., simultaneously, sequentially, or in overlapping dosing regimens) in such a manner that the subject is exposed simultaneously to both the reduced folate compound and the additional agent.

In some embodiments, the additional agent is a cofactor or coenzyme of folate or metabolite thereof. In some embodiments, the additional agent is capable of improving absorption and/or transport of folate or a reduced folate compound. In some embodiments, the additional agent acts in a pathway unrelated to folate metabolism but can supplement a deficiency in the subject and/or otherwise provide a benefit to the subject. For example, the additional agent can be an agent that is typically found in lower levels than optimal in the subject being administered the reduced folate compound. In some embodiments, the additional agent is an agent whose deficiency may be masked in the presence of higher levels of folates (e.g., vitamin B9). For example, vitamin B12 deficiencies may be masked in the presence of high vitamin B9 levels.

In some embodiments, the additional agent is a vitamin. For example, the additional agent may be vitamin B3, vitamin B12 (methylcobalamin), vitamin C, vitamin D3, or a combination thereof.

In some embodiments, the additional agent is a folate receptor (e.g., FRα) or a folate-binding fragment thereof.

Combinations of two or more additional agents may also be administered in accordance with methods of the present disclosure. For example, the two or more additional agents may comprise vitamin B12 and vitamin D3. In some embodiments, the two or more additional agents comprise vitamin B12, vitamin C, and vitamin D3.

### Compositions

In the following an exemplary composition for use in the treatment of reducing incidence or reducing risk of developing a CFD-related disorder is disclosed.

A composition comprises the components, a folate, e.g. the calcium salt of L-5-methyl-tetrahydrofolate, in an amount of 0.2 mg to 1.5 mg, and optionally one or more of the following components, N-acetylcysteine or its salt in an amount of 40 mg to 250 mg, L-selenomethionine in an amount of 0.005 mg to 0.04 mg, cholecalciferol in an amount of 0.009 mg to 0.06 mg, calcium D-panthothenate in an amount of 1 mg to 8 mg, methylcobalamin in an amount of 0.003 mg to 0.8 mg, pyridoxal-5′-phosphate in an amount of 1 mg to 4 mg, riboflavin in an amount of 2 mg to 14 mg, thiamine mononitrate in an amount of 0.2 mg to 2 mg, zeaxanthin in an amount of 1 mg to 3 mg, lutein in an amount of 4 mg to 15 mg, D-α-tocopherol in an amount of 1 mg to 8 mg, calcium ascorbate in an amount of 20 mg to 65 mg, copper gluconate in an amount of 0.1 to 1 mg and zinc acetate in an amount of 2 mg to 33 mg.

In a preferred embodiment, the components are present in the following amounts, folate, e.g. calcium salt of L-5-methyl-tetrahydrofolate, in an amount of 0.5 mg to 1.1 mg, and optionally one or more of the following components, N-acetylcysteine in an amount of 90 mg to 190 mg, L-selenomethionine in an amount of 0.01 mg to 0.03 mg, cholecalciferol in an amount of 0.015 mg to 0.045 mg, calcium D-panthothenate in an amount of 2 mg to 6 mg, methylcobalamin in an amount of 0.005 mg to 0.6 mg, pyridoxal-5′-phosphate in an amount of 1.6mg to 3.5mg, riboflavin in an amount of 3.7 mg to 10.5 mg, thiamine mononitrate in an amount of 0.45 mg to 1.6 mg, zeaxanthin in an amount of 1.9 mg to 2.1 mg, lutein in an amount of 9 mg to 11 mg, D-α-tocopherol in an amount of 2 mg to 6 mg, calcium ascorbate in an amount of 35 mg to 50 mg, copper gluconate in an amount of 0.2 mg to 0.8 mg and zinc oxide in an amount of 4 mg to 26 mg.

In another embodiment, the composition comprises the components in the following amount, folate, e.g. calcium salt of L-5-methyl-tetrahydrofolate in an amount of 0.9 mg, and optionally one or more of the following components, N-acetylcysteine in an amount of 180 mg, L-selenomethionine in an amount of 0.02 mg, cholecalciferol in an amount of 0.0375 mg, calcium D-panthothenate in an amount of 5 mg, methylcobalamin 0.5 mg, pyridoxal 5′-phosphate in an amount of 3 mg, riboflavin in an amount of 10 mg, thiamine mononitrate in an amount of 1.5 mg, zeaxanthin in an amount of 2 mg, lutein in an amount of 10 mg, D-α-tocopherol in an amount of 5 mg, calcium ascorbate in an amount of 45 mg, copper gluconate in an amount of 0.667 mg and zinc oxide in an amount of 25 mg.

In another preferred embodiment, the composition comprises the components in the following amounts, folate, e.g. calcium salt of L-5-methyl-tetrahydrofolate in an amount of 0.6 mg, and optionally one or more of the following components, N-acetylcysteine in an amount of 100 mg, L-selenomethionine in an amount of 0.02 mg, cholecalciferol in an amount of 0.02 mg, calcium D-panthothenate in an amount of 3 mg, methylcobalamin 0. 009 mg, pyridoxal 5′-phosphate in an amount of 2.1 mg, riboflavin in an amount of 4.2 mg, thiamine mononitrate in an amount of 0.55 mg, zeaxanthin in an amount of 2 mg, lutein in an amount of 10 mg, D-α-tocopherol in an amount of 3 mg, calcium ascorbate in an amount of 40 mg, copper gluconate in an amount of 0.1 mg and zinc oxide in an amount of 5 mg.

The compositions may further comprise a pharmaceutically acceptable carrier.

Further, other folate salts may be used as components of the described compositions. Such other salts are for instance a magnesium salt of folate, a sodium salt of folate, and a zinc salt of folate. Mixtures of these salts are also conceivable, so that a composition comprises two or more different folate salts, e.g., the calcium folate salt and the magnesium folate salt.

### Administration

Reduced folate compounds and/or additional agents as described herein may be formulated into any of a variety of dosage forms suitable for administration to the intended subject. In many embodiments, reduced folate compounds and/or additional agents are administered by a systemic route, e.g., orally or intranasally. For example, reduced folate compounds and/or additional agents may be formulated as a food additive or as a tablet for oral consumption. In some embodiments, tablets comprise reduced folate compounds and/or additional agents in lyophilized form. As another example, reduced folate compounds and/or additional agents may be formulated as an aerosol (e.g., as aerosolized particles), which may be inhaled by a subject to achieve intranasal delivery.

### Kits

Provided kits generally comprise (1) one or more pharmaceutically acceptable dosage form comprising a reduced folate compound; and (2) instructions for use according to a method disclosed herein. Such instructions may include, for example, information regarding one or more of the following: the subjects intended to be administered the one or more pharmaceutically acceptable forms and/or the recommended dosing regimen(s).

The one or more pharmaceutically acceptable dosage forms may also include one or more additional agents as disclosed herein, such as vitamin B12 (methylcobalamin); vitamin D3; vitamin C; a combination of vitamin B12 and vitamin D3; or a combination vitamin B12, vitamin C, and vitamin D3.

In one embodiment a method is disclosed wherein the method of reducing incidence or reducing risk of developing cerebral folate deficiency (CFD) or a CFD-related disorder, the method comprising the step of:

administering an effective amount of a reduced folate compound to a woman of childbearing age, wherein
(a) the woman or a sexual partner of the woman has a family history of a CFD-related disorder,
(b) one or more FRα autoantibodies has been detected in a fluid sample from the woman or a sexual partner of the woman, and/or
(c) the woman or a sexual partner of the woman (i) has a mutation in the methylenetetrahydrofolate reductase (MTHFR) gene or (ii) has a proximate family member with a mutation in the MTHFR gene

In another embodiment a method is disclosed wherein method of reducing incidence of cerebral folate deficiency (CFD) or a CFD-related disorder, the method comprising the step of:

administering an effective amount of a reduced folate compound to a woman of childbearing age, wherein:
(d) the woman has not been determined to be pregnant; and
(e) (i) the woman or a sexual partner of the woman has a family history of a CFD-related disorder;
   (ii) one or more FRα autoantibodies has been detected in a fluid sample from the woman or a sexual partner of the woman; or
   (iii) the woman or a sexual partner of the woman
   (1) has a mutation in the methylenetetrahydrofolate reductase (MTHFR) gene or
   (2) has a proximate family member with a mutation in the MTHFR gene.

In a further embodiment a method is described wherein said method of reducing risk of a fetus developing cerebral folate deficiency (CFD) or a CFD-related disorder, the method comprising the step of:

administering an effective amount of a reduced folate compound to a pregnant woman bearing the fetus, wherein the pregnant woman, the fetus, or the fetus's biological father has a family history of a CFD-related disorder.

In another embodiment one or more FRα autoantibodies has been detected in a fluid sample from the pregnant woman, the fetus, or the fetus's biological father.

In a further embodiment of the method the fetus or the pregnant woman (i) has a mutation in the methylenetetrahydrofolate reductase (MTHFR) gene or (ii) has a proximate family member with a mutation in the MTHFR gene.

In another embodiment a method is disclosed wherein the method of reducing risk of a child developing cerebral folate deficiency (CFD) or a CFD-related disorder, the method comprising the step of:

administering an effective amount of a reduced folate compound to a woman who is breastfeeding the child.

In another embodiment the child or a biological parent of the child has a family history of a CFD-related disorder.

In yet a further embodiment one or more FRα autoantibodies has been detected in a fluid sample from the child or a biological parent of the child.

In a further embodiment the child (i) has a mutation in the methylenetetrahydrofolate reductase (MTHFR) gene or (ii) has a proximate family member with a mutation in the MTHFR gene.

In a further embodiment a method is disclosed wherein the method of reducing risk of a subject developing cerebral folate deficiency (CFD) or a CFD-related disorder, the method comprising the step of:

administering an effective amount of a reduced folate compound to the subject, wherein the subject has a family history of a CFD-related disorder.

In another embodiment one or more FRα autoantibodies has been detected in a fluid sample from the subject, the subject's biological mother or the subject's biological father.

In a further embodiment the subject (i) has a mutation in the methylenetetrahydrofolate reductase (MTHFR) gene or (ii) has a proximate family member with a mutation in the MTHFR gene.

In another embodiment the subject is a child.

In a further embodiment the child is less than 3 years of age, less than 2.5 years of age, less than 2 years of age, less than 1.5 years of age, less than 1 year or age, or less than 6 months of age.

In another embodiment the method further comprises coadministering an effective amount of one or more additional agents selected from vitamin B3, vitamin B12 (methylcobalamin), vitamin D3, and a subunit of FRα.

In a further embodiment the method further comprises co-administering an effective amount of vitamin B12 and an effective amount of vitamin D3.

In another embodiment the CFD-related disorder is selected from the group consisting of autism spectrum disorder, obsessive compulsive disorder, attention deficit/hyperactivity disorder, and depression.

In yet another embodiment the fluid sample comprises a blood sample.

In a further embodiment the blood sample is a plasma or serum sample.

In another embodiment the blood sample is a whole blood sample or a cellular fraction of a whole blood sample.

In yet a further embodiment the fluid sample comprises an amniotic fluid, ascites, cerebrospinal fluid, lymph, sweat, urine, tears, saliva, pleural fluid, pericardial fluid, cavity rinse, or organ rinse sample.

In another embodiment the reduced folate compound is selected from the group consisting of folinic acid, methyltetrahydrofolate (MTHF), folinic acid calcium salt, leucovorin, levo-leucovorin calcium, a mixture of dextra-leucovorin and levo-leucovorin, and combinations thereof.

In a further embodiment the reduced folate compound is folinic acid or MTHF.

In another embodiment the step of administering comprises oral administration.

In a further embodiment the reduced folate compound is administered as a food additive or a tablet.

In a further embodiment the tablet comprises the reduced folate compound in lyophilized form.

In another embodiment the step of administering comprises administering the reduced folate compound intranasally.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

1. A composition for use in the treatment of reducing incidence or reducing risk of developing cerebral folate deficiency (CFD) or a CFD-related disorder, the method comprising the step of:
administering the composition comprising an effective amount of a reduced folate compound to a woman of childbearing age, wherein
(a) the woman or a sexual partner of the woman has a family history of a CFD-related disorder,
(b) one or more FRα autoantibodies has been detected in a fluid sample from the woman or a sexual partner of the woman, and/or
(c) the woman or a sexual partner of the woman (i) has a mutation in the methylenetetrahydrofolate reductase (MTHFR) gene or (ii) has a proximate family member with a mutation in the MTHFR gene

2. A composition for use in the treatment of reducing incidence or reducing risk of developing a CFD-related disorder of reducing incidence of cerebral folate deficiency (CFD) or a CFD-related disorder, the method comprising the step of:
administering the composition comprising an effective amount of a reduced folate compound to a woman of childbearing age, wherein:
(a) the woman has not been determined to be pregnant; and
(b) (i) the woman or a sexual partner of the woman has a family history of a CFD-related disorder;
(ii) one or more FRα autoantibodies has been detected in a fluid sample from the woman or a sexual partner of the woman; or
(iii) the woman or a sexual partner of the woman
(1) has a mutation in the methylenetetrahydrofolate reductase (MTHFR) gene or
(2) has a proximate family member with a mutation in the MTHFR gene.

3. A composition for use in the treatment of reducing incidence or reducing risk of developing a CFD-related disorder of reducing risk of a fetus developing cerebral folate deficiency (CFD) or a CFD-related disorder, the method comprising the step of:
administering the composition comprising an effective amount of a reduced folate compound to a pregnant woman bearing the fetus, wherein the pregnant woman, the fetus, or the fetus's biological father has a family history of a CFD-related disorder.

4. The composition for use in the treatment of reducing incidence or reducing risk of developing a CFD-related disorder of claim 3, wherein one or more FRα autoantibodies has been detected in a fluid sample from the pregnant woman, the fetus, or the fetus's biological father.

5. The composition for use in the treatment of reducing incidence or reducing risk of developing a CFD-related disorder of claim 3 or 4, wherein the fetus or the pregnant woman (i) has a mutation in the methylenetetrahydrofolate reductase (MTHFR) gene or (ii) has a proximate family member with a mutation in the MTHFR gene.

6. A composition for use in the treatment of reducing incidence or reducing risk of developing a CFD-related disorder of reducing risk of a child developing cerebral folate deficiency (CFD) or a CFD-related disorder, the method comprising the step of:
administering a composition comprising an effective amount of a reduced folate compound to a woman who is breastfeeding the child.

7. The composition for use in the treatment of reducing incidence or reducing risk of developing a CFD-related disorder of claim 6, wherein the child or a biological parent of the child has a family history of a CFD-related disorder.

8. The composition for use in the treatment of reducing incidence or reducing risk of developing a CFD-related disorder of claim 6 or 7, wherein one or more FRα autoantibodies has been detected in a fluid sample from the child or a biological parent of the child.

9. The composition for use in the treatment of reducing incidence or reducing risk of developing a CFD-related disorder of any one of claims 6-8, wherein the child (i) has a mutation in the methylenetetrahydrofolate reductase (MTHFR) gene or (ii) has a proximate family member with a mutation in the MTHFR gene.

10. A composition for use in the treatment of reducing incidence or reducing risk of developing a CFD-related disorder of reducing risk of a subject developing cerebral folate deficiency (CFD) or a CFD-related disorder, the method comprising the step of:
administering composition comprising an effective amount of a reduced folate compound to the subject, wherein
the subject has a family history of a CFD-related disorder.

11. The composition for use in the treatment of reducing incidence or reducing risk of developing a CFD-related disorder of claim 10, wherein one or more FRα autoantibodies has been detected in a fluid sample from the subject, the subject's biological mother or the subject's biological father.

12. The composition for use in the treatment of reducing incidence or reducing risk of developing a CFD-related disorder of claim 10 or 11, wherein the subject (i) has a mutation in the methylenetetrahydrofolate reductase (MTHFR) gene or (ii) has a proximate family member with a mutation in the MTHFR gene.

13. The composition for use in the treatment of reducing incidence or reducing risk of developing a CFD-related disorder of claim 10, 11, or 12, wherein the subject is a child.

14. The composition for use in the treatment of reducing incidence or reducing risk of developing a CFD-related disorder of claim 13, wherein the child is less than 3 years of age, less than 2.5 years of age, less than 2 years of age, less than 1.5 years of age, less than 1 year or age, or less than 6 months of age.

15. The composition for use in the treatment of reducing incidence or reducing risk of developing a CFD-related disorder of any one of claims 1-14, wherein the method further comprises coadministering a composition comprising an effective amount of one or more additional agents selected from vitamin B3, vitamin B12 (methylcobalamin), vitamin D3, and a subunit of FRα.

16. The composition for use in the treatment of reducing incidence or reducing risk of developing a CFD-related disorder of claim 15, wherein the method further comprises co-administering composition comprising an effective amount of vitamin B12 and an effective amount of vitamin D3.

17. The composition for use in the treatment of reducing incidence or reducing risk of developing a CFD-related disorder of any one of claims 1-16, wherein the CFD-related disorder is selected from the group consisting of autism spectrum disorder, obsessive compulsive disorder, attention deficit/hyperactivity disorder, and depression.

18. The composition for use in the treatment of reducing incidence or reducing risk of developing a CFD-related disorder of any one of claim 1, 2, 4, 8, or 11, wherein the fluid sample comprises a blood sample.

19. The composition for use in the treatment of reducing incidence or reducing risk of developing a CFD-related disorder of claim 18, wherein the blood sample is a plasma or serum sample.

20. The composition for use in the treatment of reducing incidence or reducing risk of developing a CFD-related disorder of claim 18, wherein the blood sample is a whole blood sample or a cellular fraction of a whole blood sample.

21. The composition for use in the treatment of reducing incidence or reducing risk of developing a CFD-related disorder of any one of claim 1, 2, 4, 8, 11, or 18-20, wherein the fluid sample comprises an amniotic fluid, ascites, cerebrospinal fluid, lymph, sweat, urine, tears, saliva, pleural fluid, pericardial fluid, cavity rinse, or organ rinse sample.

22. The composition for use in the treatment of reducing incidence or reducing risk of developing a CFD-related disorder of any one of claims 1-21, wherein the reduced folate compound is selected from the group consisting of folinic acid, methyltetrahydrofolate (MTHF), folinic acid calcium salt, leucovorin, levo-leucovorin calcium, a mixture of dextra-leucovorin and levo-leucovorin, and combinations thereof.

23. The composition for use in the treatment of reducing incidence or reducing risk of developing a CFD-related disorder of claim 22, wherein the reduced folate compound is folinic acid or MTHF.

24. The composition for use in the treatment of reducing incidence or reducing risk of developing a CFD-related disorder of any one of claims 1-23, wherein the step of administering comprises oral administration.

25. The composition for use in the treatment of reducing incidence or reducing risk of developing a CFD-related disorder of claim 24, wherein the reduced folate compound is administered as a food additive or a tablet.

26. The composition for use in the treatment of reducing incidence or reducing risk of developing a CFD-related disorder of claim 25, wherein the tablet comprises the reduced folate compound in lyophilized form.

27. The composition for use in the treatment of reducing incidence or reducing risk of developing a CFD-related disorder of any one of claims 1-26, wherein the step of administering comprises administering the reduced folate compound intranasally.

28. A kit, comprising a composition for use in the treatment of reducing incidence or reducing risk of developing a CFD-related disorder comprising one or more pharmaceutically acceptable dosage forms, wherein at least one pharmaceutically acceptable dosage form comprises a reduced folate compound, and instructions for use according to any one of claims 1-27.

29. The kit of claim 28, wherein the composition for use in the treatment of reducing incidence or reducing risk of developing a CFD-related disorder comprising one or more pharmaceutically dosage forms collectively comprise one or more additional agents selected from the group consisting of vitamin B12 (methylcobalamin) and vitamin D3.

30. The kit of claim 29, wherein the composition for use in the treatment of reducing incidence or reducing risk of developing a CFD-related disorder comprising one or more pharmaceutically dosage forms collectively comprise vitamin B12 and vitamin D3.
